(19) United States
(12) Patent Application Publication
(10) Pub. No.: US 2004/0229878 A1
(43) Pub. Date: Nov. 18, 2004

(54) Transdiscal administration of specific inhibitors of P38 kinase

(75) Inventors: Thomas M. DiMauro, Southboro, MA (US); Hassan Serhan, South Easton, MA (US); Mohamed Attawia, Canton, MA (US); Melissa Grace, Raynham, MA (US); Sudhakar Kadiyala, South Easton, MA (US); David Urbahns, Barrington, RI (US); Scott Bruder, Sudbury, MA (US); Gregory Collins, East Sandwich, MA (US); Laura J. Brown, Hamilton Square, NJ (US); Jeff Geesin, Doylestown, PA (US); Pamela L. Plouhar, South Bend, IN (US); Catherine Smith, East Falmouth, MA (US); John Siekierka, Towaco, NJ (US)

Correspondence Address: HAMILTON, BROOK, SMITH & REYNOLDS, P.C., 530 VIRGINIA ROAD, P.O. BOX 9133, CONCORD, MA, 01742-9133, US

(73) Assignee: DePuy Spine, Inc., Raynham, MA (US)

(21) Appl. No.: 10/631,487

(22) Filed: Jul. 31, 2003

Related U.S. Application Data
(63) Continuation-in-part of application No. 10/610,355, filed on Jun. 30, 2003.
Application No. 10/610,355 is a continuation-in-part of application No. 10/456,948, filed on Jun. 6, 2003.
(60) Provisional application No. 60/470,098, filed on May 13, 2003.

(51) Int. Cl. A61K 31/503; A61K 31/519; A61K 31/415; A61K 31/12; A61K 31/17

(52) U.S. Cl. 514/248; 514/305; 514/406; 514/396; 514/419; 514/596; 514/686; 514/262.1

(57) ABSTRACT

The present invention relates to injecting a high specificity p38 kinase inhibitor into a diseased intervertebral disc.

### RELATED APPLICATIONS

This application is a continuation-in-part of U.S. application Ser. No. 10/610,355, filed Jun. 30, 2003, which is a continuation-in-part application of U.S. patent application Ser. No. 10/456,948, DiMauro et al., filed Jun. 6, 2003 and which claims priority from U.S. Provisional Application No. 60/470,098, filed May 13, 2003. The entire teachings of the above applications are incorporated herein by reference.

### BACKGROUND OF THE INVENTION

The natural intervertebral disc contains a jelly-like nucleus pulposus surrounded by a fibrous annulus fibrosus. Under an axial load, the nucleus pulposus compresses and radially transfers that load to the annulus fibrosus. The laminated nature of the annulus fibrosus provides it with a high tensile strength and so allows it to expand radially in response to this transferred load.

In a healthy intervertebral disc, cells within the nucleus pulposus produce an extracellular matrix (ECM) containing a high percentage of proteoglycans. These proteoglycans contain sulfated functional groups that retain water, thereby providing the nucleus pulposus with its cushioning qualities. These nucleus pulposus cells may also secrete small amounts of cytokines as well as matrix metalloproteinases (“MMPs”). These cytokines and MMPs help regulate the metabolism of the nucleus pulposus cells.

In some instances of disc degeneration disease (DDD), gradual degeneration of the intervertebral disc is caused by mechanical instabilities in other portions of the spine. In these instances, increased loads and pressures on the nucleus pulposus cause the cells to emit larger than normal amounts of the above-mentioned cytokines. In other instances of DDD, genetic factors, such as programmed cell death, or apoptosis can also cause the cells within the nucleus pulposus to emit toxic amounts of these cytokines and MMPs. In some instances, the pumping action of the disc may malfunction (due to, for example, a decrease in the proteoglycan concentration within the nucleus pulposus), thereby retarding the flow of nutrients into the disc as well as the flow of waste products out of the disc. This reduced capacity to eliminate waste may result in the accumulation of high levels of toxins.

As DDD progresses, the toxic levels of the cytokines present in the nucleus pulposus begin to degrade the extracellular matrix. In particular, the MMPs (under mediation by the cytokines) begin cleaving the water-retaining portions of the proteoglycans, thereby reducing their water-retaining capabilities. This degradation leads to a less flexible nucleus pulposus, and so changes the load pattern within the disc, thereby possibly causing delamination of the annulus fibrosus. These changes cause more mechanical instability, thereby causing the cells to emit even more cytokines, typically thereby upregulating MMPs. As this destructive cascade continues and DDD further progresses, the disc begins to bulge (“a herniated disc”), and then ultimately ruptures, causing the nucleus pulposus to contact the spinal cord and produce pain.

Olmarker, Spine 26(8): 863-9 (2001) (“Olmarker I”) and Aoki, Spine 27(15): 1614-17 (2002), teach that TNF-α appears to play a role in producing the pain associated with the nucleus pulposus contacting nerve roots of the spinal cord.

U.S. Published patent application No. 2003/0039651 (“Olmarker II”) teaches a therapeutic treatment of nerve disorders comprising administration of a therapeutically effective dosage of at least two substances selected from the group consisting of TNF inhibitors (both specific and non-specific), IL-1 inhibitors, IL-6 inhibitors, IL-8 inhibitors, FAS inhibitors, FAS ligand inhibitors, and IFN-gamma inhibitors.

In the examples of Olmarker II, Olmarker II further teaches that these substances are to be administered through systemic pathways. In particular, Olmarker II teaches that “the major contribution of TNF-alpha may be derived from recruited, aggregated and maybe even extravasated leukocytes, and that successful pharmacologic block may be achieved only by systemic treatment. Of note, Olmarker II appears to discourage the local addition of one therapeutic agent (doxycycline) to a transplanted nucleus pulposus.

PCT Published Patent Application No. WO 02/100387 (“Olmarker III”) teaches the prevention of neovascularization and/or neo-innervation of intervertebral discs by the administration of anti-angiogenic substances. Again, however, Olmarker III teaches systemic administration of these therapeutic agents.

U.S. Pat. No. 6,419,944 (“Tobinick”) discloses treating herniated discs with cytokine antagonists, including REMICADE® infliximab. Tobinick teaches that local administration involves a subcutaneous injection near the spinal cord. Accordingly, Tobinick does not teach a procedure involving a sustained delivery of a drug for the treatment of DDD, nor directly administering a specific cytokine antagonist (such as infliximab) into the disc.

U.S. Published patent application No. 2003/0049256 (Tobinick II) discloses that injection of such therapeutic molecules to the anatomic area adjacent to the spine is accomplished by interspinous injection, and preferably is accomplished by injection through the skin in the anatomic area between two adjacent spinous processes of the vertebral column.

Tobinick II further teaches that TNF antagonists may be administered by interspinous injection in the human and that the dosage level is in the range of 1 mg to 300 mg per dose, with dosage intervals as short as two days. Tobinick II further discloses that Interleukin-1 antagonists are administered in a therapeutically effective dose, which will generally be 10 mg to 200 mg per dose, and their dosage interval will be as short as once daily.

Tobinick, Swiss Med. Weekly, 133:170-77 (2003), (“Tobinick III”) teaches both perispinal and epidural administration of TNF inhibitors for spine related therapies.

Karppinen, Spine, 28(8):750-4 (2003), teaches intravenously injecting or orally administering infliximab into patients suffering from sciatica.

As with Tobinick I and II, Karppinen does not teach a procedure involving a sustained delivery of a drug for the treatment of DDD, nor directly administering a specific cytokine antagonist (such as REMICADE® infliximab) into the disc.

U.S. Pat. No. 6,352,557 (Ferree) teaches adding therapeutic substances such as anti-inflammatory medications to morselized extra-cellular matrix, and injecting that combination into an interverterbral disc.

However many anti-inflammatory agents are non-specific and therefore may produce unwanted side effects upon other cells, proteins and tissue. In addition, the pain-reducing effect of these agents is typically only temporary. Lastly, these agents typically only relieve pain, and are neither curative nor restorative.

Alini, Eur. Spine J. 11(Supp.2):S215-220 (2002), teaches therapies for early stage DDD, including injection of inhibitors of proteolytic enzymes or biological factors that stimulate cell metabolic activity (i.e., growth factors) in order to slow down the degenerative process. Alini I does not disclose inhibiting growth factors.

U.S. Published patent application No. 2002/0026244 (“Trieu”) discloses an intervertebral disc nucleus comprising a hydrogel that may deliver desired pharmacological agents. Trieu teaches that these pharmacological agents may include growth factors such as TGF-B and anti-inflammatory drugs, including steroids. Trieu further teaches that these pharmacological agents may be dispersed within the hydrogel having an appropriate level of porosity to release the pharmacological agent at a desired rate. Trieu teaches that these agents may be released upon cyclic loading or upon resorption. Takegami, Spine, 27(12):1318-25 (2000) teaches that injecting TGF-B into the disc space results in enhanced replenishment of the extracellular matrix damaged by cytokines. Takegami further teaches that the half-life of a growth factor injected into the interveterbal disc can be expected to be longer than that injected into a synovial joint because the nucleus pulposus is surrounded by the fibrous structure of the annulus fibrosus, thus providing a confined environment. Diwan, Tissue Engineering in Orthopedic Surgery, 31(3):453-464 (2000), reports on another Takegami paper that concluded that a delivery system allowing prolonged delivery (>3 days) would have to be used to obtain the observed effect of the growth factor.

Alini, Spine 28(5):446-54 (2003), discloses a cell seeded collagen-hyaluronan scaffold supplemented with growth factors such as TGF-B, bFGF, and IGF-1 for use in regenerating a nucleus pulposus.

Maeda et al. Spine 25(2):166-169 (2000) reports on the in vitro response to interleukin-1 receptor antagonist protein (IRAP) of rabbit annulus fibrosus exposed to IL-1. Maeda suggests that IRAP could be useful in inhibiting the degradation of the disc.

Yabuki, Spine, 26(8):870-5 (2001), teaches the use of an anti-TNF drug for the treatment of sciatica.

U.S. Pat. No. 6,277,969 (“Le”) discloses the use of anti-TNF antibodies for therapy of TNF-mediated pathologies. Le teaches parenteral administration of the antibodies.

Ariga, Spine, 28(14):1528-33 (2003), reports that the application of a p38 MAP kinase inhibitor to a culture of organ-cultured intervertebral discs increased the occurrence of apoptosis in endplate chondrocytes in the culture.

### SUMMARY OF THE INVENTION

The present inventors have developed a number of procedures for efficaciously treating degenerative disc disease by drug therapy.

In accordance with the present invention, the present inventors have developed a method of treating an intervertebral disc in which a high specificity inhibitor of a pro-inflammatory cytokine is administered transdiscally (i.e., the target tissue is a degenerating disc).

There are several advantages to directly administering these therapeutic inhibitors to a targeted disc:

First, since it is known that many cytokines (such as interleukins and TNF-α) also play roles in mediating the degradation of the extracellular matrix (ECM) of the nucleus pulposus, injecting an antagonist or inhibitor of these proteins directly into the disc prevents the target cytokine from inducing any further ECM degradation. In effect, the transdiscal adminstration of the cytokine antagonist arrests the aging process of the degenerating disc. Accordingly, the present invention seeks to treat the degenerative disc at a much earlier stage of DDD thereby prevents degradation of the ECM.

Second, it is further known that nerve ending nociceptors are present within the annulus fibrosus, and that cytokines such as TNF irritate nerves. It is believed that injecting an anti-TNF antagonist into the disc space also prevents the TNF from causing nerve irritation within the disc. Thus, the pain attributed to irritation of these nerves can be efficiently eliminated.

Third, since the annulus fibrosus portion of the disc comprises a relatively dense fibrosus structure, this outer component of the disc may provide a suitable depot for the high specificity cytokine antagonist (HSCA), thereby increasing its half-life in the disc.

Fourth, since the high specificity antagonist inhibits only the specific cytokine of interest, the HSCA may be combined with other therapeutic agents (such as TGF-β or mesenchymal stem cells) that can also be injected into the disc without reducing the effectiveness of those agents.

Fifth, since it is believed that many of the problematic cytokines are actually secreted by either nucleus pulposus or annulus fibrosus cells, transdiscal injection of the high specificity antagonists will advantageously attack the problematic cytokines at their source of origination.

Accordingly, in a first aspect of the present invention, there is provided a method of treating an intervertebral disc having a nucleus pulposus, comprising transdiscally administering a formulation comprising a high specificity cytokine antagonist (HSCA) into an intervertebral disc.

### DETAILED DESCRIPTION OF THE INVENTION

A description of preferred embodiments of the invention follows.

For the purposes of the present invention, the terms “inhibitor” and antagonist” are used interchangeably. A protein may be inhibited at the synthesis level, at the translation level, by shedding, by antibodies, or by soluble receptors. The term “patient” refers to a human having a degenerating disc.

For the purposes of the present invention “Transdiscal administration” includes, but is not limited to:

a) injecting a formulation into the nucleus pulposus of a degenerating disc, such as a relatively intact degenerating disc,

b) injecting a formulation into the annulus fibrosus of a degenerating disc, such as relatively intact degenerating disc,

c) providing a formulation in a patch attached to an outer wall of the annulus fibrosus,

d) providing a formulation in a depot at a location outside but closely closely adjacent to an outer wall of the annulus fibrosus (“trans-annular administration”), and

e) providing the formulation in a depot at a location outside but closely adjacent to an endplate of an adjacent vertebral body (“trans-endplate administration”).

Because DDD is a continuous process, the degenerating disc to which the therapeutic drug is administered may be in any one of a number of degenerative states. Accordingly, the degenerating disc may be an intact disc. The degenerating disc may be a herniated disc (wherein a portion of the annulus fibrosus has a bulge). The degenerating disc may be a ruptured disc (wherein the annulus fibrosus has ruptured and bulk nucleus pulposus has exuded). The degenerating disc may be delaminated (wherein adjacent layers of the annulus fibrosus have separated). The degenerating disc may have fissures (wherein the annulus fibrosus has fine cracks or tears through which selected molecules from the nucleus pulposus can leak).

The present invention is directed to providing directly through a diseased intervertebral disc at least one highly specific cytokine antagonist capable of specifically inhibiting a cytokine (preferably, a pro-inflammatory cytokine) present in the disc. In one embodiment, the HSCA inhibits the action of a specific pro-inflammatory cytokine released by disc cells or by invading macrophages during the degenerative process.

In some embodiments, the antagonist is capable of specifically inhibiting a pro-inflammatory cytokine selected from the group consisting of TNF-α, an interleukin (preferably, IL-1, Il-6 and IL-8), FAS, an FAS ligand, and IFN-gamma. Such specific inhibitors include those identified on pages 5-18 of Olnarker II, the specification of which is incorporated herein by reference in its entirety.

In some embodiments, the HSCA inhibits the cytokine by preventing its production. In some embodiments, the HSCA inhibits the cytokine by binding to a membrane-bound cytokine. In others, the HSCA inhibits the cytokine by binding to a solubilized, e.g. soluble, cytokine. In some embodiments, the HSCA inhibitor inhibits the cytokine by both binding to membrane bound cytokines and to solubilized cytokines. In some embodiments, the HSCA is a monoclonal antibody (“mAb”). The use of mAbs is highly desirable since they bind specifically to a certain target protein and to no other proteins. In some embodiments, the HSCA inhibits the cytokine by binding to a natural receptor of the target cytokine.

In some embodiments, the HSCA inhibits the cytokine by preventing its production. One example thereof is an inhibitor of p38 mitogen activated protein (MAP) kinase. In some embodiments, the TNF inhibitor inhibits the TNF by binding to membrane-bound TNF in order to prevent its release from membrane. In others, the TNF inhibitor inhibits the TNF by binding to solubilized TNF. One example thereof is etanercept. In some embodiments, the TNF inhibitor inhibits the TNF by both binding to membrane bound TNF and to solubilized TNF. One example thereof is REMICADE® infliximab. In some embodiments, the HSCA inhibits the cytokine (e.g., TNF-α) by binding to a natural receptor of the target cytokine. In some embodiments, the TNF-α inhibitor is an inhibitor of TNF-α synthesis.

Preferred TNF antagonists include, but are not limited to, the following: etanercept (ENBREL® Amgen); infliximab (REMICADE®.-Johnson and Johnson); D2E7, a human anti-TNF monoclonal antibody (Knoll Pharmaceuticals, Abbott Laboratories); CDP 571 (a humanized anti-TNF IgG4 antibody); CDP 870 (an anti-TNF alpha humanized monoclonal antibody fragment), both from Celltech; soluble TNF receptor Type I (Amgen); pegylated soluble TNF receptor Type I (PEGs TNF-R1) (Amgen); and onercept, a recombinant TNF binding protein (r-TBP-1) (Serono).

TNF antagonists suitable for compositions, combination therapy, co-administration, devices and/or methods of the present invention (optionally further comprising at least one antibody, specified portion and/or variant thereof, of the present invention), include, but are not limited to, anti-TNF antibodies (e.g., at least one TNF antagonist (e.g., but not limited to a TNF chemical or protein antagonist, TNF monoclonal or polyclonal antibody or fragment, a soluble TNF receptor (e.g., p55, p70 or p85) or fragment, fusion polypeptides thereof, or a small molecule TNF antagonist, e.g., TNF binding protein I or II (TBP-1 or TBP-II), nerelimonmab, REMICADE® infliximab, enteracept (ENBREL®), adalimulab (HUMIRA™), CDP-571, CDP-870, afelimomab, lenercept, and the like), antigen-binding fragments thereof, and receptor molecules which bind specifically to TNF; compounds which prevent and/or inhibit TNF synthesis, TNF release or its action on target cells, such as thalidomide, tenidap, phosphodiesterase inhibitors (e.g. pentoxifylline and rolipram), A2b adenosine receptor agonists and A2b adenosine receptor enhancers; compounds which prevent and/or inhibit TNF receptor signalling, such as mitogen activated protein (MAP) kinase inhibitors; compounds which block and/or inhibit membrane TNF cleavage, such as metalloproteinase inhibitors; compounds which block and/or inhibit TNF activity, such as angiotensin converting enzyme (ACE) inhibitors (e.g., captopril); and compounds which block and/or inhibit TNF production and/or synthesis, such as MAP kinase inhibitors.

As used herein, a “tumor necrosis factor antibody,” “TNF antibody,” “TNFα antibody,” or fragment and the like decreases, blocks, inhibits, abrogates or interferes with TNFα activity in vitro, in situ and/or preferably in vivo. For example, a suitable TNF human antibody of the present invention can bind TNFα and includes anti-TNF antibodies, antigen-binding fragments thereof, and specified mutants or domains thereof that bind specifically to TNF-alpha (TNFα). A suitable TNF antibody or fragment can also decrease block, abrogate, interfere, prevent and/or inhibit TNF RNA, DNA or protein synthesis, TNF release, TNF receptor signaling, membrane TNF cleavage, TNF activity, TNF production and/or synthesis.

Chimeric antibody cA2 consists of the antigen binding variable region of the high-specificity neutralizing mouse anti-human TNFα IgG1 antibody, designated A2, and the constant regions of a human IgG1, kappa immunoglobulin. The human IgG1 Fc region improves allogeneic antibody effector function, increases the circulating serum half-life and decreases the immunogenicity of the antibody. The avidity and epitope specificity of the chimeric antibody cA2 is derived from the variable region of the murine antibody A2. In a particular embodiment, a preferred source for nucleic acids encoding the variable region of the murine antibody A2 is the A2 hybridoma cell line.

Chimeric A2 (cA2) neutralizes the cytotoxic effect of both natural and recombinant human TNFα in a dose dependent manner. From binding assays of chimeric antibody cA2 and recombinant human TNFα, the specificity constant of chimeric antibody cA2 was calculated to be 1.04×10¹⁰M⁻¹. Preferred methods for determining monoclonal antibody specificity and specificity by competitive inhibition can be found in Harlow et al., Antibodies: A Laboratory Manual, Cold Spring Harbor Laboratory Press, Cold Spring Harbor, N.Y. (1988); Colligan et al., eds., Current Protocols in Immunology, Greene Publishing Assoc. and Wiley Interscience, New York, (1992-2000); Kozbor et al., Immunol. Today, 4:72-79 (1983); Ausubel et al., eds. Current Protocols in Molecular Biology, Wiley lnterscience, New York (1987-2000); and Muller, Meth. Enzymol., 92:589-601 (1983), which references are entirely incorporated herein by reference.

In a particular embodiment, murine monoclonal antibody A2 is produced by a cell line designated c134A. Chimeric antibody cA2 is produced by a cell line designated c168A.

Additional examples of monoclonal anti-TNF antibodies that can be used in the present invention are described in the art (see, e.g., U.S. Pat. No. 5,231,024; Möller, A. et al., Cytokine 2(3): 162-169 (1990); U.S. application Ser. No. 07/943,852 (filed Sep. 11, 1992); Rathjen et al., International Publication No. WO 91/02078 (published Feb. 21, 1991); Rubin et al., EPO Patent Publication No. 0 218 868 (published Apr. 22, 1987); Yone et al., EPO Patent Publication No. 0 288 088 (Oct. 26, 1988); Liang, et al., Biochem. Biophys. Res. Comm. 137:847-854 (1986); Meager, et al., Hybridoma 6:305-311 (1987); Fendly et al., Hybridoma 6:359-369 (1987); Bringman, et al., Hybridoma 6:489-507 (1987); and Hirai, et al., J. Immunol. Meth. 96:57-62 (1987), which references are entirely incorporated herein by reference).

Preferred TNF receptor molecules useful in the present invention are those that bind TNF α with high specificity (see, e.g., Feldmann et al., International Publication No. WO 92/07076 (published Apr. 30, 1992); Schall et al., Cell, 61:361-370 (1990); and Loetscher et al., Cell 61:351-359 (1990), which references are entirely incorporated herein by reference) and, optionally, possess low immunogenicity. In particular, the 55 kDa (p55 TNF-R) and the 75 kDa (p75 TNF-R) TNF cell surface receptors are useful in the present invention. Truncated forms of these receptors, comprising the extracellular domains (ECD) of the receptors or functional portions thereof (see, e.g., Corcoran et al., Eur. J. Biochem. 223:831-840 (1994)), are also useful in the present invention. Truncated forms of the TNF receptors, comprising the ECD, have been detected in urine and serum as 30 kDa and 40 kDa TNFα inhibitory binding proteins (Engelmann, H. et al., J. Biol. Chem. 265:1531-1536 (1990)). TNF receptor multimeric molecules and TNF immunoreceptor fusion molecules, and derivatives and fragments or portions thereof, are additional examples of TNF receptor molecules which are useful in the methods and compositions of the present invention. The TNF receptor molecules which can be used in the invention are characterized by their ability to treat patients for extended periods with good to excellent alleviation of symptoms and low toxicity. Low immunogenicity and/or high specificity, as well as other undefined properties, can contribute to the therapeutic results achieved.

TNF receptor multimeric molecules useful in the present invention comprise all or a functional portion of the ECD of two or more TNF receptors linked via one or more polypeptide linkers or other nonpeptide linkers, such as polyethylene glycol (PEG). The multimeric molecules can further comprise a signal peptide of a secreted protein to direct expression of the multimeric molecule. These multimeric molecules and methods for their production have been described in U.S. application Ser. No. 08/437,533 (filed May 9, 1995), the content of which is entirely incorporated herein by reference.

TNF immunoreceptor fusion molecules useful in the methods and compositions of the present invention comprise at least one portion of one or more immunoglobulin molecules and all or a functional portion of one or more TNF receptors. These immunoreceptor fusion molecules can be assembled as monomers, or hetero- or homo-multimers. The immunoreceptor fusion molecules can also be monovalent or multivalent. An example of such a TNF immunoreceptor fusion molecule is TNF receptor/IgG fusion protein. TNF immunoreceptor fusion molecules and methods for their production have been described in the art (Lesslauer et al., Eur. J. Immunol. 21:2883-2886 (1991); Ashkenazi et al., Proc. Natl. Acad. Sci. USA 88:10535-10539 (1991); Peppel et al., J. Exp. Med. 174:1483-1489 (1991); Kolls et al., Proc. Natl. Acad. Sci. USA 91:215-219 (1994); Butler et al., Cytokine 6(6):616-623 (1994); Baker et al., Eur. J. Immunol. 24:2040-2048 (1994); Beutler et al., U.S. Pat. No. 5,447,851; and U.S. application Ser. No. 08/442,133 (filed May 16, 1995), each of which references are entirely incorporated herein by reference). Methods for producing immunoreceptor fusion molecules can also be found in Capon et al., U.S. Pat. No. 5,116,964; Capon et al., U.S. Pat. No. 5,225,538; and Capon et al., Nature 337:525-531 (1989), which references are entirely incorporated herein by reference.

A functional equivalent, derivative, fragment or region of a TNF receptor molecule refers to the portion of the TNF receptor molecule, or the portion of the TNF receptor molecule sequence which encodes the TNF receptor molecule, that is of sufficient size and sequences to functionally resemble TNF receptor molecules that can be used in the present invention (e.g., bind TNFα with high specificity and possess low immunogenicity). A functional equivalent of a TNF receptor molecule also includes modified TNF receptor molecules that functionally resemble TNF receptor molecules that can be used in the present invention (e.g., bind TNFα with high specificity and possess low immunogenicity). For example, a functional equivalent of a TNF receptor molecule can contain a “SILENT” codon or one or more amino acid substitutions, deletions or additions (e.g., substitution of one acidic amino acid for another acidic amino acid; or substitution of one codon encoding the same or different hydrophobic amino acid for another codon encoding a hydrophobic amino acid). See Ausubel et al., Current Protocols in Molecular Biology, Greene Publishing Assoc. and Wiley-Interscience, New York (1987-2003).

In some embodiments, the monoclonal antibody that inhibits TNF-α is selected from the group consisting of monoclonal rodent-human antibodies, rodent antibodies, human antibodies or any portions thereof, having at least one antigen binding region of an immunoglobulin variable region, which antibody binds TNF. Preferably, this monoclonal antibody is selected from the group of compounds disclosed in U.S. Pat. No. 6,277,969, the specification of which is entirely incorporated by reference. In some embodiments, the REMICADE® infliximab is delivered in a formulation having an infliximab concentration of between about 30 mg/ml and about 60 mg/ml.

In some embodiments, the specific inhibitor of TNF-α is an inhibitor of p38 MAP kinase, preferably, a small molecule inhibitor of p38 MAP kinase. The inhibition of p38 MAP kinase is believed to block production of both TNF-α and Il-2, both of which are pro-inflammatory cytokines. The small molecule inhibitors of p38 MAP kinase are very specific & potent (˜nM). Without wishing to be tied to a theory, it is believed that inhibition of p38 should not block TGF signaling nor TGF activity. It is further believed that p38 inhibitors may also block induction of some metalloproteinases, COX 2 and NO synthetase. It is further believed that P38 inhibitors do not inhibit interleukins involved in immune cell proliferation such as IL-2.

In some embodiments, the HSCA is a specific antagonist of an interleukin. Preferably, the target interleukin is selected from the group consisting IL-1, IL-2, IL-6 and IL-8, and IL-12. Preferred antagonists include but are not limited to Kineretg (recombinant IL 1-RA, Amgen), IL1-Receptor Type 2 (Amgen) and IL-1 Trap (Regeneron).

DDD involves the progressive degeneration of a disc in which many factors are involved. In many instances, simply providing a single dose or even a regimen over the space of a few days may not be sufficient to resolve the DDD. For example, if DDD were caused in part by mechanical instability in a functional spinal unit, then simply providing a one-time therapy for the disc cells would likely only delay the onset of the DDD. Therefore, there is a need to provide a long-term drug therapy treatment of DDD that does not require multiple injections.

Because the cytokines of interest both produce pain and degrade the ECM when present within the nucleus pulposus, it is desirable for the HSCA to remain within the nucleus pulposus as long as possible in a pharmaceutically effective amount. The half-life of the HSCA within the nucleus pulposus will depend upon many factors, including the size of the HSCA and its charge. In general, the larger the molecular weight of the HSCA, the more likely it is to remain contained by the annulus fibrosus portion of the disc.

If the half-life of the HSCA is relatively short, then it would be desirable for a relatively large dose of the HSCA to be administered into the disc. In this condition, quick depletion of the HSCA would not cause the HSCA to fall below therapeutically effective levels until an extended period.

Although a large dose of the HSCA would be desirable in such instances, injecting a critical volume of water can increase pressure in the nucleus pulposus. Nociceptors present on the inner wall of the annulus fibrosus react to this increased pressure and produce pain. One avenue for increasing the pressure in the nucleus pulposus is to inject a critical volume of water. In some cases, the added amount could be as little as one cc by volume to produce pain. Accordingly, if a dilute concentration of an HSCA is added to the nucleus pulposus to provide a large dose, the resulting pressure increase caused by this added volume could be sufficient to cause acute pain.

For example, if it were determined that about 100 mg of an HSCA was needed to therapeutically affect a nucleus pulposus, and that HSCA was provided in concentrations of from about 30-60 mg/ml, then at least about 1.5 ml of the HSCA should be injected into the nucleus pulposus in order to provide the desired therapeutic effect. However, when injecting volumes into the nucleus pulposus, it is desirable that the volume of drug delivered be no more than about 1 ml, preferably no more than 0.5 ml, (i.e., a maximum of about 0.5 ml) more preferably between about 0.1 and about 0.3 ml. When injected in these smaller quantities, it is believed the added volume will not cause an appreciable pressure increase in the nucleus pulposus.

Accordingly, in some embodiments, the concentration of the TNF-α antagonist in the administered drug is at least about 100 mg/ml. When 100 mg of the HSCA is needed to produce the desired therapeutic result, no more than about 1 ml of the drug need be injected. Preferably, the concentration of the TNF-α antagonist in the administered drug is at least 200 mg/ml. In this condition, no more than about 0.5 ml of the drug need be injected. Preferably, the concentration of the TNF-α antagonist in the administered drug is at least 500 mg/ml. In this condition, between about 0.03 ml and about 0.3 ml of the drug need be injected.

In some embodiments, the HSCA is provided in a sustained release device. The sustained release device is adapted to remain within the disc for a prolonged period and slowly release the HSCA contained therein to the surrounding environment. This mode of delivery allows an HSCA to remain in therapeutically effective amounts within the disc for a prolonged period.

In some embodiments, the HSCA is predominantly released from the sustained delivery device by its diffusion through the sustained delivery device (preferably, though a polymer). In others, the HSCA is predominantly released from the sustained delivery device by the biodegradation of the sustained delivery device (preferably, biodegradation of a polymer).

Preferably, the sustained release device (i.e., sustained delivery device) comprises a bioresorbable material whose gradual erosion causes the gradual release of the HSCA to the disc environment. In some embodiments, the sustained release device comprises a bioresorbable polymer. Preferably, the bioresorbable polymer has a half-life of at least one month, more preferably at least two months, more preferably at least 6 months.

In some embodiments, the sustained release device provides controlled release. In others, it provides continuous release. In others, it provides intermittent release. In others, the sustained release device comprises a biosensor.

In some embodiments, the sustained delivery device comprises a plurality of bioerodable macrospheres. The HSCA is preferably contained in a gelatin (or water or other solvent) within the capsule, and is released to the disc environment when the outer shell has been eroded. The device can include a plurality of capsules having outer shells of varying thickness, so that the sequential breakdown of the outer shells provides periodic release of the HSCA.

In some embodiments, the sustained delivery device comprises an inflammatory-responsive delivery system, such as one comprising bioerodable microspheres that are eroded by invading macrophages. This technology provides a high correspondence between physiologic inflammation of disc environment and the release of the HSCAs into that environment. Preferably, the technology disclosed in Brown et al., Arthritis. Rheum.; 41(12): 2185-95 (Dec. 1998) is selected.

In some embodiments, the sustained delivery device comprises the devices disclosed in U.S. Pat. No. 5,728,396 (“Peery”), the specification of which is incorporated by reference in its entirety.

In some embodiments, the sustained delivery device comprises a plurality (e.g., at least one hundred) of water-containing chambers, each chamber containing the HSCA. Each chamber is defined by bilayer lipid membranes comprising synthetic duplicates of naturally occurring lipids. The release of the drug can be controlled by varying at least one of the aqueous excipients, the lipid components, and the manufacturing parameters. Preferably, the formulation comprises no more than 10% lipid. In some embodiments, the DEPOFOAM™ technology of Skyepharma PLC (London, United Kingdom) is selected.

In some embodiments, the sustained delivery device comprises a delivery system disclosed in U.S. Pat. No. 5,270,300 (“Hunziker”), the specification of which is incorporated by reference in its entirety.

In some embodiments, the sustained delivery device comprises the co-polymer poly-DL-lactide-co-glycolide (PLG). Preferably, the formulation is manufactured by combining the HSCA, the co-polymer and a solvent to form a droplet, and then evaporating the solvent to form a microsphere. The plurality of microspheres are then combined in a biocompatible diluent. Preferably, the HSCA is released from the co-polymer by its diffusion therethrough and by the biodegradation of the co-polymer. In some embodiments hereof, the ProLease™ technology of Alkermes (Cambridge, Mass.) is selected.

In some embodiments, the sustained delivery device comprises a hydrogel. Hydrogels can also be used to deliver the HSCA in a time-release manner to the disc environment. A “hydrogel” is a substance formed when an organic polymer (natural or synthetic) is set or solidified to create a three-dimensional open-lattice structure that entraps molecules of water or other solution to form a gel. The solidification can occur, e.g., by aggregation, coagulation, hydrophobic interactions, or cross-linking. The hydrogels employed in this invention rapidly solidify to keep the HSCA at the application site, thereby eliminating undesired migration from the disc. The hydrogels are also biocompatible, e.g., not toxic, to cells suspended in the hydrogel.

A “hydrogel-HSCA composition” is a suspension of a hydrogel containing desired HSCA. The hydrogel-HSCA composition forms a uniform distribution of HSCA with a well-defined and precisely controllable density. Moreover, the hydrogel can support very large densities of HSCA. In addition, the hydrogel allows diffusion of nutrients and waste products to, and away from, the HSCA, which promotes tissue growth.

Hydrogels suitable for use in the present invention include water-containing gels, i.e., polymers characterized by hydrophilicity and insolubility in water. See, for instance, “Hydrogels”, pages 458-459, in Concise Encyclopedia of polymer Science and Engineering, Eds. Mark et al., Wiley and Sons, (1990), the disclosure of which is incorporated herein entirely by reference. Although their use is optional in the present invention, the inclusion of hydrogels can be highly advantageous since they tend to contribute a number of desirable qualities. By virtue of their hydrophilic, water-containing nature, hydrogels can:

a) house viable cells, such as mesenchymal stems cells, and

b) assist with load bearing capabilities of the disc.

In a preferred embodiment, the hydrogel is a fine, powdery synthetic hydrogel. Suitable hydrogels exhibit an optimal combination of such properties as compatibility with the matrix polymer of choice, and biocompatability. The hydrogel can include one or more of the following: polysaccharides, proteins, polyphosphazenes, poly(oxyethylene)-poly(oxypropylene) block polymers, poly(oxyethylene)-poly(oxypropylene) block polymers of ethylene diamine, poly(acrylic acids), poly(methacrylic acids), copolymers of acrylic acid and methacrylic acid, poly(vinyl acetate), and sulfonated polymers.

In general, these polymers are at least partially soluble in aqueous solutions, e.g., water, or aqueous alcohol solutions that have charged side groups, or a monovalent ionic salt thereof. There are many examples of polymers with acidic side groups that can be reacted with cations, e.g., poly(phosphazenes), poly(acrylic acids), and poly(methacrylic acids). Examples of acidic groups include carboxylic acid groups, sulfonic acid groups, and halogenated (preferably fluorinated) alcohol groups. Examples of polymers with basic side groups that can react with anions are poly(vinyl amines), poly(vinyl pyridine), and poly(vinyl imidazole).

In some embodiments, the sustained delivery device includes a polymer selected from the group consisting of PLA, PGA, PCL and mixtures thereof.

If the half-life of the HSCA within the disc is relatively long, then it may be assumed that a relatively small dose of the HSCA can be administered into the disc. In this condition, the slow depletion of the HSCA would not cause the HSCA to fall below therapeutically effective levels in the disc until an extended period of time has elapsed.

In some embodiments in which HSCAs have long half-lives within the disc, the dose administered can be very small.

For example, if it is believed that an HSCA is effective when present in the range of about 1-10 mg/kg or 1-10 ppm (as is believed to be the case for the TNF antagonist REMICADE® infliximab), and since a typical nucleus pulposus of a disc has a volume of about 3 ml (or 3 cc, or 3 g), then only about 3-30 μg of the HSCA need be administered to the disc in order to provide a long lasting effective amount of the drug. As a point of reference, Tobinick discloses that at least 1 mg of cytokine antagonist should be administered perispinally in order to cure back pain. Similarly, Olmarker mixed 100 ml of a formulation comprising 1.11 mg/ml of a monoclonal antibody into 40 mg of an extracted nucleus pulposus, thereby producing a monoclonal antibody concentration of about 3 parts per thousand. The smaller amounts available by this route reduce the chances of deleterious side effects of the HSCA.

For example, suppose a clinician administered 0.3 ml of 60 mg/ml REMICADE® infliximab into a 2.7 cc disc, thereby producing an infliximab concentration in the disc of about 6 mg/ml, or 6 parts per thousand. Without wishing to be tied to a theory, if infliximab has the same half-life within a nucleus pulposus as it does when administered systemically (i.e., about 1 week), then the concentration of infliximab would remain above about 10 ppm for about 9 weeks. Therefore, if another dose were needed, the clinician would only need to provide the second dose after about two months.

Therefore, in some embodiments, the HSCA is provided in a dose of less than about 1 mg, for example, a maximum of about 0.5 mg, e.g., less than about 0.5 mg, more preferably, less than about 0.1 mg, more preferably less than about 0.01 mg, e.g., less than about 0.001 mg. The smaller amounts available by this route reduce the chances of deleterious side effects of the HSCA. Preferably, the HSCA provided in these smaller amounts is a TNF antagonist, more preferably it is REMICADE® infliximab. The formulation is administered in an amount effective to reduce pain.

In some embodiments, the formulation is administered in an effective amount. In some embodiments, the formulation is administered in an amount effective to reduce pain. In some embodiments, the formulation is administered in an amount effective to inhibit degradation of the ECM of the nucleus pulposus.

In preferred embodiments, the formulation of the present invention is administered directly into the disc through the outer wall of the annulus fibrosus. In one embodiment, the direct administration includes depositing the HSCA in the nucleus pulposus portion of the disc. In this condition, the fibrous nature of the annulus fibrosus that surrounds the nucleus pulposus will help keep the HSCA contained within the disc.

Preferably, the formulation of the present invention is injected into the disc through a small bore needle. More preferably, the needle has a bore of about 22 gauge or less, so that the possibilities of producing a herniation are mitigated. For example, the needle can have a bore of about 24 gauge or less, so that the possibilities of producing a herniation are even further mitigated.

If the volume of the direct injection of the formulation is sufficiently high so as to cause a concern of overpressurizing the nucleus pulposus, then it is preferred that at least a portion of the nucleus pulposus be removed prior to direct injection. In some embodiments, the volume of removed nucleus pulposus is substantially similar to the volume of the formulation to be injected. For example, the volume of removed nucleus pulposus can be within about 80-120% of the volume of the formulation to be injected.

In addition, this procedure has the added benefit of at least partially removing some degenerated disc from the patient.

In other embodiments, the formulation is delivered into the disc space through the endplate of an opposing vertebral body. This avenue eliminates the need to puncture the annulus fibrosus, and so eliminates the possibility of herniation.

Although the cytokine antagonists may therapeutically treat the disc by binding the target cytokine, thereby reducing pain and arresting degradation of the ECM, it is believed that at least some of these antagonists do not help repair the damage done by the cytokine to the ECM.

Therefore, there may be a need to provide a therapy that also helps repair the ECM.

In accordance with the present invention, there is provided a method of treating degenerative disc disease in an intervertebral disc having a nucleus pulposus, comprising:

a) administering a highly specific cytokine antagonist into a degenerating disc; and

b) administering at least one additional therapeutic agent in an amount effective to at least partially repair the disc.

In accordance with one aspect of the invention, both the HSCA and additional (e.g., second) therapeutic agent(s) are locally administered into the disc. Because the HSCA is specific, it does not interfere with the locally administered additional therapeutic agent, and so each agent may independently work to provide therapy to the diseased disc.

More than one additional therapeutic agent can be administered. For example, there can be third, fourth and fifth therapeutic agents.

In some embodiments, the HSCA and additional therapeutic agent(s) are administered simultaneously. In others, the HSCA is administered first. In still others, the additional therapeutic agent(s) is/are administered first.

Other compounds which may be added to the disc include, but are not limited to: vitamins and other nutritional supplements; hormones; glycoproteins; fibronectin; peptides and proteins; carbohydrates (both simple and/or complex); proteoglycans; oligonucleotides (sense and/or antisense DNA and/or RNA); bone morphogenic proteins (BMPs); antibodies (for example, to infectious agents, tumors, drugs or hormones); gene therapy reagents and anti-cancer agents. Genetically altered cells and/or other cells may also be included in the matrix of this invention. If desired, substances such as pain killers and narcotics may also be admixed with a polymer for delivery and release to the disc space.

In one embodiment, healthy cells are introduced into the disc that have the capability of at least partially repairing any damage done to the disc during the degenerative process. In some embodiments, these cells are introduced into the nucleus pulposus and ultimately produce new extracellular matrix for the nucleus pulposus. In others, these cells are introduced into the annulus fibrosus and produce new extracellular matrix for the annulus fibrosus.

In some embodiments, these cells are obtained from another human individual (allograft), while in others embodiments, the cells are obtained from the same individual (autograft). In some embodiments, the cells are taken from an intervertebral disc (and can be either nucleus pulposus cells or annulus fibrosus cells), while in others, the cells are taken from a non-disc tissue (and may be mesenchymal stem cells). In others, autograft chondrocytes (such as from the knee, hip, shoulder, finger or ear) may be used.

Preferably, when viable cells are selected as an additional therapeutic agent or substance, the viable cells comprise mesenchymal stem cells (MSCs). MSCs provide a special advantage for administration into a degenerating disc because it is believed that they can more readily survive the relatively harsh environment present in the degenerating disc; that they have a desirable level of plasticity; and that they have the ability to proliferate and differentiate into the desired cells.

In some embodiments, the mesenchymal stem cells are obtained from bone marrow, preferably autologous bone marrow. In others, the mesenchymal stem cells are obtained from adipose tissue, preferably autologous adipose tissue.

In some embodiments, the mesenchymal stem cells injected into the disc are provided in an unconcentrated form. In others, they are provided in a concentrated form. When provided in concentrated form, they can be uncultured. Uncultured, concentrated MSCs can be readily obtained by centrifugation, filtration, or immuno-absorption. When filtration is selected, the methods disclosed in U.S. Pat. No. 6,049,026 (“Muschler”), the specification of which is incorporated by reference in its entirety, are preferably used. In some embodiments, the matrix used to filter and concentrate the MSCs is also administered into the nucleus pulposus. If this matrix has suitable mechanical properties, it can be used to restore the height of the disc space that was lost during the degradation process.

In some embodiments, growth factors are additional therapeutic agents. As used herein, the term “growth factors” encompasses any cellular product that modulates the growth or differentiation of other cells, particularly connective tissue progenitor cells. The growth factors that may be used in accordance with the present invention include, but are not limited to, members of the fibroblast growth factor family, including acidic and basic fibroblast growth factor (FGF-1 and FGF-2) and FGF-4; members of the platelet-derived growth factor (PDGF) family, including PDGF-AB, PDGF-BB and PDGF-AA; EGFs; members of the insulin-like growth factor (IGF) family, including IGF-I and -II; the TGF-β superfamily, including TGF-p1, 2 and 3 (including MP-52); osteoid-inducing factor (OIF), angiogenin(s); endothelins; hepatocyte growth factor and keratinocyte growth factor; members of the bone morphogenetic proteins (BMP's) BMP-1, BMP-3; BMP-2; OP-1; BMP-2A, BMP-2B, and BMP-7, BMP-14; HBGF-1 and HBGF-2; growth differentiation factors (GDF's), members of the hedgehog family of proteins, including indian, sonic and desert hedgehog; ADMP-1; members of the interleukin (IL) family, including IL-1 thru IL-6; GDF-5 and members of the colony-stimulating factor (CSF) family, including CSF-1, G-CSF, and GM-CSF; and isoforms thereof.

In some embodiments, the growth factor is selected from the group consisting of TGF-β, bFGF, and IGF-1. These growth factors are believed to promote regeneration of the nucleus pulposus. Preferably, the growth factor is TGF-β. More preferably, TGF-β is administered in an amount of between about 10 ng/ml and about 5000 ng/ml, more preferably between about 50 ng/ml and about 500 ng/ml, more preferably between about 100 ng/ml and about 300 ng/ml.

In some embodiments, platelet concentrate is provided as an additional therapeutic agent. In one embodiment, the growth factors released by the platelets are present in an amount at least two-fold (e.g., four-fold) greater than the amount found in the blood from which the platelets were taken. In some embodiments, the platelet concentrate is autologous. In some embodiments, the platelet concentrate is platelet rich plasma (PRP). PRP is advantageous because it contains growth factors that can restimulate the growth of the ECM, and because its fibrin matrix provides a suitable scaffold for new tissue growth.

Since it is known that many pro-inflammatory proteins play a role in disc degeneration, and that the antagonists of the present invention are highly specific, it is further believed that injecting at least two of the highly specific antagonists of the present invention directly into the disc would be advantageous.

Therefore, in accordance with the present invention, there is provided a method of treating degenerative disc disease in an intervertebral disc having a nucleus pulposus, comprising administering a formulation comprising at least two highly specific antagonists of pro-inflammatory cytokines selected from the group consisting of TNF-α, an interleukin (preferably, IL-1, Il-6 and IL-8), FAS, an FAS ligand, and IFN-gamma.

Preferably, at least one of the substances is an antagonist of TNF-α. Preferably, the other substance is an antagonist of an interleukin.

In some embodiments, the formulation comprises a suitable biocompatible carrier such as saline. In some embodiments, the carrier is selected from the carriers disclosed in U.S. Pat. No. 6,277,969 (“Le”), the specification of which is incorporated by reference in its entirety. In some embodiments, the formulation includes a solvent, preferably selected from the group consisting of DMSO and ethanol.

In some embodiments, the formulation is administered through a drug pump.

Also in accordance with the present invention, there is provided a formulation for treating degenerative disc disease, comprising:

a) a high specificity cytokine antagonist, and

b) an additional therapeutic agent selected from the group consisting of:

i) a growth factor, and

ii) viable cells.

In some embodiments of this formulation, the high specificity cytokine antagonist is selected from the group consisting of antagonists of TNF and antagonists of an interleukin.

Because the causes of low back pain may be myriad, and because of the significant cost of many of these specialized HSCAs, it would be useful for the clinician to first perform a diagnostic test in order to confirm that the targeted disc in fact possesses high levels of the targeted cytokine prior to providing the injection.

In one embodiment, the diagnostic test comprises a non-invasive diagnostic test comprising using magnetic resonance imaging (MRI).

Preferably, the clinician would first perform a discogram in order to identify which disc or discs are responsible for the patient's low back pain. Next, the clinician would perform an invasive or non-invasive test upon the targeted disc in order to confirm the presence of or quantify the level of the pro-inflammatory cytokine.

In one embodiment, the diagnostic test comprises an invasive test in which a portion of the disc is removed and analysed. In some embodiments, the clinician removes a portion of the nucleus pulposus. In some embodiments, the clinician removes a portion of the annulus fibrosus. Preferably, the removed material is a portion of the nucleus pulposus. The presence of pro-inflammatory cytokines in the removed material may detected by procedures including, but not limited, to electrophoresis, or an enzyme-linked immunoabsorbent assay (ELISA) (as per Burke, Br. JBJS, 84-B(2), 2002).

In some embodiments, the diagnostic methods disclosed in U.S. Pat. No. 6,277,969 (“Le”), the specification of which is incorporated by reference in its entirety, are selected. In these methods, high specificity anti- TNF-α compounds are used as diagnostic tools for detecting TNF-α in the patient known or suspected to have a high level of TNF-α.

After determining the levels of the different pro-inflammatory cytokines in the degenerating disc, the clinician will preferably proceed to compare these diagnosed levels against pre-determined levels of the pro-inflammatory cytokines. If the diagnosed level of the pro-inflammatory cytokine exceeds the pre-determined level, then the clinician may conclude that these higher levels are causing unwanted inflammatory action and proceed to directly inject a specific HSCA (e.g., a high specificity p38 kinase inhibitor) into the disc capable of inhibiting the targeted protein.

In some embodiments, the predetermined level for an interleukin is at least 100 pg/ml. In some embodiments, the predetermined level for IL-6 is at least 250 pg/ml. In some embodiments, the predetermined level for IL-8 is at least 500 pg/ml. In some embodiments, the predetermined level for PGE2 is at least 1000 pg/ml. In some embodiments, the predetermined level for TNF-α is at least 500 pg/ml. In others, the predetermined level for TNF-α is at least 20 pg/ml, more preferably at least 30 pg/ml, more preferably at least 50 pg/ml, more preferably at least 1 ng/ml. In others, the predetermined level for TNF-α is at least 1 ng/disc, and in others, it is at least 1000 pg/disc.

It would also be useful to be able to determine whether directly administering the therapeutic substances of the present invention was, in fact, efficacious. Accordingly, one can measure the level of cytokine remaining in the disc after administration.

The present invention can also be used to prevent degeneration of an intervertebral disc in a human individual, namely, by following a procedure comprising the steps of:

a) determining a genetic profile of the individual,

b) comparing the profile of the individual against a pre-determined genetic profile level of at-risk humans,

c) determining that the individual is an at-risk patient, and

d) injecting an antagonist of the pro-inflammatory protein into a disc of the individual.

Transdiscal administration of an effective amount of other high specificity antagonists of pro-inflammatory processes can also help provide therapy to the patient having degenerative disc disease (DDD). In many embodiments, the high specificity antagonist is a high specificity antagonist of an enzyme, such as an enzyme of a kinase.

Transdiscal administration of an effective amount of a high specificity antagonist of p38 kinase would also help provide therapy to a patient having DDD. It is believed that the p38 kinase site regulates the production of TNF-α, IL-1 and COX-2 enzyme.

Therefore, in accordance with another embodiment of the present invention, there is provided a method of treating degenerative disc disease in an intervertebral disc having a nucleus pulposus, comprising transdiscally administering an effective amount of a formulation comprising a high specificity antagonist of p38 kinase into an intervertebral disc.

Accordingly, in some embodiments, the highly specific antagonist is an inhibitor of p38 MAP kinase, preferably, a small molecule inhibitor of p38 MAP kinase. The inhibition of p38 MAP kinase is believed to block production of both TNF-α and Il-2, both of which are pro-inflammatory cytokines. The small molecule inhibitors of p38 MAP kinase are very specific and potent (˜nM). Without wishing to be tied to a theory, it is believed that inhibition of p38 should not block TGF signaling nor TGF activity. It is further believed that p38 inhibitors may also block induction of some metalloproteinases, COX 2 and NO synthetase. It is further believed that P38 inhibitors do not inhibit interleukins involved in immune cell proliferation such as IL-2. Preferably, they are provided in a 10 nM to 10 uM dose. Some high specificity antagonists of p38 kinase are disclosed in Zhang, J. Biol. Chem., 272(20): 13397-402 (May, 16 1997); Pargellis, Nature Structural Biology, 9(4): 268-272 (April 2002); and Chae, Bone, 28(1): 45-53 (January 2001), and in U.S. Pat. No. 6,541,477 (“Goehring”) and U.S. Pat. No. 5,965,583 (Beers), the specifications of which are hereby incorporated by reference in their entirety. Preferably, the HSA of p38 kinase is administered in a dosage to produce a local tissue concentration of between about 5 ug/kg and 50 ug/kg.

In some embodiments, the p38 kinase inhibitor is selected from the group consisting of:

a) diaryl imidizole;

b) N,N′-diaryl urea (developed by Bayer, Boehringer Ingelheim and Vertex);

c) N,N-diaryl urea (developed by Vertex);

d) benzophenone (developed by Leo Pharmaceuticals);

e) pyrazole ketone (developed by Hoffmnan-LaRoche);

f) indole amide (developed by GlaxoSmithKline and Scios);

g) diamides (developed by AstraZeneca);

h) quinazoline (developed by GlaxoSmithKline);

i) pyrimido [4,5-d]pyrimidinone (developed by GlaxoSmithKline and Hoffman LaRoche);

j) pyridylamino-quinazolines (developed by Scios).

Members of this group are described, for example, in Zhang et al., supra, Pargellis et al., supra, Chae et al., supra, and Cirillo et al., Current Topics in Medicinal Chemistry, 2: 1021-1035 (2002), Boehm et al., Exp. Opin, Ther. Patents, 10(1):25-38 (2000), and Lee et al., Immunopharmacology, 47: 185-2001 (2000).

In some embodiments, the p38 kinase inhibitor is selected from the group consisting of:

a) SK&F 86002;

b) SB 203580;

c) L-167307;

d) HEP 689;

e) SB220025;

f) VX-745;

g) SU4984;

h) RWJ 68354;

i) ZM336372;

j) PD098059;

k) SB235699; and

l) SB220025.

In some embodiments, the p38 kinase inhibitor is characterized as a 1-aryl-2-pyridinyl heterocycle. In some embodiments, the 1-aryl-2-pyridinyl heterocycle is selected from the group consisting of:

a) 4,5 substituted imidazole,

b) 1,4,5 substitutued imidizole;

c) 2,4,5 substututued imidizole;

d) 1,2,4,5 substituted imidizole; and

e) non-imidizole 5-membered ring heterocycle.

In some embodiments, the p38 kinase inhibitor has at least 3 cyclic groups.

In some embodiments, the p38 kinase inhibitor is selected from the group consisting of a molecule that is readily soluble in water and a substantially water insoluble molecule. In some embodiments, the highly specific antagonist is a p38 kinase inhibitor that is a substantially water insoluble molecule. The substantially water insoluble p38 inhibitor may be advantageous in that, if injected into the nucleus pulposus, it will remain in the nucleus pulposus as a solid and only slightly solubize over time, thereby providing sustained release.

It is further believed that transdiscal administration of an effective amount of a high specificity antagonist of the COX-2 enzyme would also help provide therapy to the patient having DDD. It is believed that the COX-2 enzyme is a regulator of the production of prostaglandins, which are involved both in inflammation and the generation of pain.

Therefore, in accordance with another embodiment of the present invention, there is provided a method of treating degenerative disc disease in an intervertebral disc having a nucleus pulposus, comprising transdiscally administering an effective amount of a formulation comprising a high specificity antagonist of COX-2 enzyme into an intervertebral disc.

Typical high specificity antagonists of the COX-2 enzyme include Celecoxib (Searle), Rofecoxib (Merck), Meloxican (Boehringer Manheim), Nimesulide, diclofenac and Lodine.

It is further believed that transdiscal administration of an effective amount of a high specificity antagonist of the NO synthase enzyme would also help provide therapy to the patient having DDD. It is believed that the NO synthase enzyme regulates the production of NO, which is known to have pro-inflammatory effects.

Therefore, in accordance with another embodiment of the present invention, there is provided a method of treating degenerative disc disease in an intervertebral disc having a nucleus pulposus, comprising transdiscally administering an effective amount of a formulation comprising a high specificity antagonist of NO synthase into an intervertebral disc.

Examples of high specificity antagonists include NO synthase are N-iminoethyl-L-lysine (L-NIL), and N^{G}-monomethyl-L-arginine.

In some embodiments, the high specificity antagonists of NO synthase may be administered systemically.

It is further believed that transdiscal administration of an effective amount of a high specificity anti-oxidant would also help provide therapy to the patient having DDD. It is believed that oxidants degrade the nucleus pulposus extra-cellular matrix. Typical anti-oxidants include free radical scavengers and superoxide dismutase enzymes.

Therefore, in accordance with another embodiment of the present invention, there is provided a method of treating degenerative disc disease in an intervertebral disc having a nucleus pulposus, comprising transdiscally administering an effective amount of a formulation comprising a high specificity antioxidant into an intervertebral disc.

In some embodiments, the high specificity antioxidants may be administered systemically.

It is further believed that trandiscal administration of an effective amount of a high specificity anti-proliferative agent would also help provide therapy to the patient having DDD. It is believed that antiproliferative agents may have an effect on inflammation by affecting inflamed tissues which would limit the production of inflammatory cytokines. In some embodiments, the high specificity anti-proliferative is selected from the group consisting of a) rapamycin; b) an inhibitor of cyclin dependent kinase 9 (cdk); and c) statins (such as MEVASTATIN™ and LOVASTATIN™). In one embodiment, when rapamycin is selected, a dosage producing a local tissue concentration of between about 0.5 ug/kg and 50 ug/kg is used.

Therefore, in accordance with another embodiment of the present invention, there is provided a method of treating degenerative disc disease in an intervertebral disc having a nucleus pulposus, comprising transdiscally administering an effective amount of a formulation comprising a high specificity anti-proliferative agent into an intervertebral disc.

Rapamycin is a potent inhibitor of downstream signaling of TOR (target of Rapamycin) proteins. As such, it is responsible for coordinating the balance between protein synthesis and protein degradation. It is believed that DDD is propagated by a loss of balance between extracellular matrix synthesis and degradation. Since TOR proteins regulate multiple metabolic pathways, rapamycin may stabilize the balance of the cycle. Rapamycin may also directly affect the proliferation and subsequent immune reaction of chondrocytes. In addition, degenerative chondrocytes demonstrate a low level of proliferative activity in contrast to normal chondrocytes which show no activity. This is thought to lead to chondrocyte clustering within the cartilage. Rapamycin could function to eliminate the atypical chondrocyte proliferation. In one embodiment, it is provided in a about 0.1 to about 10 μM dose.

Cdk inhibitors may directly affect the proliferation and subsequent immune reaction of chondrocytes. A cdk inhibitor may also have a direct effect on chondrocyte clustering which is known to be a characteristic degenerative event. Exemplary cdk inhibitors include flavopiridol, roscovitine, and compounds disclosed in PCT Patent Publication No. WO 02/057240 (Lin) and U.S. provisional patent application 60/257,703, the specifications of which are incorporated by reference herein in their entirety. In one embodiment, it is provided in a 1 to 10 μM dose.

The present invention is also directed to providing a highly specific anti-apoptosis molecule to the diseased joint. These molecules serve to protect against chondrocyte apoptosis. Preferred compounds include EPO, erythropoetin mimetic peptides, EPO mimetibodies, IGF-I, IGF-II, and caspase inhibitors.

Therefore, in accordance with another embodiment of the present invention, there is provided a method of treating degenerative disc disease in an intervertebral disc having a nucleus pulposus, comprising transdiscally administering an effective amount of a formulation comprising a high specificity anti-apoptotic agent into an intervertebral disc.

In addition, non-steroidal anti-inflammatory drugs (NSAIDs) may also be selected as the second therapeutic agent. In some embodiments, the NSAID is anabolic, and is preferably selected from the group consisting of TOLMETIN™ (available from Ortho-MacNeil), SUPROL™ (available from Johnson & Johnson), and Tiaprofenic acid, (available from Roussel Labs). Preferably, the anabolic NSAID is administered in a dosage sufficient to produce an initial local tissue concentration of between about 5 ug/kg and 500 ug/kg. In some embodiments, the NSAID is a dual inhibitor of both the COX and LOX pathways, and is preferably TEPOXALIN™ (available from Johnson & Johnson).

In addition, anti-cathepsins may also be used in accordance with the present invention. It is believed that inhibition of these enzymes inhibits the breakdown of the extracellular matrix. Preferably, the antagonists inhibits a cathepsin selected from the group consisting of cathepsin B, cathepsin L and cathepsin K.

In some preferred embodiments, the antagonist is combined in the formulation with a viscosupplement. The viscosupplement has characteristics substantially similar to that of natural healthy nucleus pulposus ECM.

Preferably, the viscosupplement comprises glycosaminoglycans (GAGS). GAGS are biopolymers consisting of repeating polysaccharide units, and are present in nature on the cell surface as well as in the extracellular matrix of animals. GAGS are long unbranded polysaccharides containing a repeating disaccharide unit. The disaccharide unit contains either of two modified sugars, N-acetylgalactosaimne or N-acetylglucosamine and a uronic acid such as glucuronate or iduronate. GAGS are highly negatively charged molecules, with extended conformation that imparts high viscosity to the solution. In addition, to high viscosity, GAGS routinely possess low compressability, which makes these molecules ideal for a lubricating fluid in the joints. At the same time, their rigidity provides structural integrity to cells and provides passageways between cells, allowing for cell migration.

Hyaluronic acid (HA) is a high molecular weight polysaccharide of N-acetyl glucosaime and glucuronic acid molecules that is naturally occurring in all mammals in a variety of tissue and some bacterial species. For the purposes of this invention, HA includes any derivatives such as hyaluronan and hyaluronic acid itself with H⁺ ion attached to the COO⁻ group, and salts of hyaluronic acid whereby another positive ion replaces the H⁺ ion, as for example, with Na⁺ which forms sodium hyaluronate. Also included in the definition of HA is any physically or chemically cross-linked hyaluronic acid or derivative. HA is unique among the GAGS in that it does not contain any sulphate and is not found covalently attached to proteins as a proteoglycan. HA polymers are very large, with molecular weights of between about 100,000 and 10,000,000, and can displace a large volume of water. For the purposes of the present invention, a preferred embodiment includes a non-cross linked HA with a molecular weight of 0.5-10 M Dalton.

Preferably, the viscosupplement is selected from the group consisting of hyaluronic acid and hyaluronate (either cross-linked or uncross-linked).

In some embodiments, cartilage cells (which may be from either an allogeneic or autologous source) or mesenchymal stem cells, may be genetically modified to produce a cartilage anabolic agent which could be chosen from the list of growth factors named herein. The production of these chondroprotective agents, differentiation promoting agents would lead to tissue repair.

Recent work has shown that plasmid DNA will not elicit an inflammatory response as does the use of viral vectors. Genes encoding cartilage (anabolic) agents such as BMP may be efficacious if injected into the joint. In addition, overexpression of any of the growth factors provided herein or other agents such as TIMP which would limit local MMP activity would have positive effects on chondrocyte and ECM protection. In one embodiment, the plasmid contains the genetic code for human TGF-B or EPO.

Accordingly, in some embodiments, the additional therapeutic agent selected from the group consisting of:

i) a growth factor,

ii) viable cells, and

iii) plasmid DNA.

### EXAMPLE I

This non-limiting prophetic example describes how to transdiscally administer a formulation comprising a HSCA and saline into a nucleus pulposus of a degenerating disc.

First, a clinician uses a diagnostic test to verify that a particular disc within a patient has high levels of a particular pro-inflammatory cytokine.

Next, the clinician provides a local anesthetic (such as 5 ml lidocaine) to the region dorsal of the disc of concern to reduce subcutaneous pain.

Next, the clinician punctures the skin of the patient dorsal the disc of concern with a relatively large (e.g., 18-19 gauge) needle having a stylet therein, and advances the needle through subcutaneous fat and dorsal sacrolumbar ligament and muscles to the outer edge of the intervertebral disc.

Next, the stylet is removed from the needle.

Next, the clinician receives a syringe having a smaller gauge needle adapted to fit within the larger gauge needle. This needle is typically a 22 or 24 gauge needle. The barrel of the syringe contains the formulation of the present invention.

The formulation contains REMICADE® infliximab, and has an infliximab concentration of between about 30 mg/ml and about 60 mg/ml.

Next, the physician advances the smaller needle co-axially through the larger needle and past the distal end of the larger needle, thereby puncturing the annulus fibrosus. The smaller needle is then further advanced into the center of the nucleus pulposus. Finally, the clincian depresses the plunger of the syringe, thereby injecting between about 0.1 and 1 ml of the formulation into the nucleus pulposus.

### EXAMPLE II

This non-limiting prophetic example is substantially similar to that of Example I, except that the formulation comprises a sustained release device comprising the co-polymer poly-DL-lactide-co-glycolide (PLG). The formulation contains infliximab as the antagonist, and has an infliximab concentration of between about 30 mg/ml and about 60 mg/ml.

While this invention has been particularly shown and described with references to preferred embodiments thereof, it will be understood by those skilled in the art that various changes in form and details may be made therein without departing from the scope of the invention encompassed by the appended claims.

What is claimed is:

1. A method of treating degenerative disc disease in an intervertebral disc having a nucleus pulposus and an annulus fibrosus, comprising transdiscally administering an effective amount of a formulation comprising a high specificity antagonist of p38 kinase into an intervertebral disc.

2. The method of claim 1 wherein the p38 kinase inhibitor is selected from the group consisting of:
i) diaryl imidizole;
ii) N,N′-diaryl urea;
iii) N,N-diaryl urea;
iv) benzophenone;
v) pyrazole ketone;
vi) indole amide;
vii) diamides;
viii) quinazoline;
ix) pyrimido [4,5-d]pyrimidinone; and
x) pyridylamino-quinazolines.

3. The method of claim 1 wherein the p38 kinase inhibitor is substantially water insoluble.

4. The method of claim 1 wherein the formulation further comprises at least one additional second therapeutic agent.

5. The method of claim 1 wherein the formulation is administered in an amount effective to reduce pain.

6. The method of claim 1 wherein the formulation is administered in an amount effective to inhibit degradation of an extracellular matrix of the nucleus pulposus.

7. The method of claim 1 wherein the p38 kinase is administered in a dosage to produce a local tissue concentration of between about 5 μg/kg and 50 μg/kg.

8. The method of claim 1 wherein the p38 kinase inhibitor is water soluble.

9. The method of claim 1 wherein the the p38 kinase inhibitor is a 1-aryl-2-pyridinyl heterocycle.

10. The method of claim 1 wherein the 1-aryl-2-pyridinyl heterocycle is selected from the group consisting of:
f) 4,5 substituted imidazole;
g) 1,4,5 substitutued imidizole;
h) 2,4,5 substututued imidizole;
i) 1,2,4,5 substituted imidizole; and
non-imidizole 5-membered ring heterocycle.

11. The method of claim 1 wherein the the p38 kinase inhibitor has at least 3 cyclic groups.

12. The method of claim 1 wherein the formulation is administered in an amount of less than about 1 cc.

13. The method of claim 1 wherein the p38 kinase inhibitor is present in the formulation in an amount of at least about 100 mg/ml.

14. The method of claim 1 wherein the formulation further comprises a sustained release device.

15. The method of claim 14 wherein the sustained release device comprises a hydrogel.

16. The method of claim 14 wherein the sustained release device provides controlled release.

17. The method of claim 14 wherein the sustained release device provides continuous release.

18. The method of claim 14 wherein the sustained release device provides intermittent release.

19. The method of claim 14 wherein the sustained release device comprises a biosensor.

20. The method of claim 14 wherein the sustained release device comprises a plurality of microspheres.

21. The method of claim 14 wherein the sustained release device comprises an inflammatory-responsive delivery system.

22. The method of claim 1 wherein the formulation is provided closely adjacent the outer wall of the annulus fibrosus.

23. The method of claim 1 wherein the p38 kinase inhibitor is present in the formulation in an amount of no more than about 0.5 mg.

24. The method of claim 1 wherein the formulation further comprises a growth factor present in an amount effective to repair disc tissue.

25. The method of claim 24 wherein the growth factor is provided by platelet concentrate.

26. The method of claim 1 wherein the formulation is administered through a drug pump.

27. The method of claim 1 wherein the formulation further comprises viable mesenchymal stem cells.

28. The method of claim 1 wherein the formulation is injected into the nucleus pulposus.

29. The method of claim 1 wherein the formulation is injected into the annulus fibrosus.

30. The method of claim 1 wherein a portion of the nucleus pulposus is removed prior to transdiscally administering the formulation.

31. The method of claim 1 wherein the administration is performed through a needle.

32. The method of claim 1 wherein the formulation further comprises glycosaminoglycans.

33. The method of claim 1 wherein the formulation is administered in a volume of between about 0.03 ml and about 0.3 ml.

34. The method of claim 1 wherein the administration comprises providing the formulation in a patch attached to an outer wall of the annulus fibrosus.

35. The method of claim 1 wherein the administration comprises providing the formulation in a depot at a location closely adjacent to an outer wall of the annulus fibrosus.

36. The method of claim 1 wherein the administration comprises providing the formulation in a depot at a location closely adjacent to an endplate of an adjacent vertebral body.

37. The method of claim 1 wherein the degenerating disc is an intact disc.

38. The method of claim 1 wherein the degenerating disc is a ruptured disc.

39. The method of claim 1 wherein the degenerating disc is delaminated.

40. The method of claim 1 wherein the degenerating disc has fissures.

41. The method of claim 1 wherein the antagonist is predominantly released from the sustained delivery device by its diffusion through the sustained delivery device.

42. The method of claim 41 wherein the sustained delivery device is a polymer.

43. The method of claim 1 wherein the antagonist is predominantly released from the sustained delivery device by biodegradation of the sustained delivery device.

44. A formulation for treating degenerative disc disease, comprising:
a) a high specificity p38 kinase inhibitor, and
b) an additional therapeutic agent selected from the group consisting of:
i) a growth factor,
ii) viable cells, and
iii) plasmid DNA.

45. The formulation of claim 44 wherein the additional therapeutic agent is plasmid DNA.

46. The formulation of claim 44 wherein the additional therapeutic agent is viable cells comprising mesenchymal stem cells.

47. The formulation of claim 46 wherein the mesenchymal stem cells are autologous.

48. The formulation of claim 47 wherein the mesenchymal stem cells are provided in a concentrated form.

49. The formulation of claim 44 wherein the additional therapeutic agent is a growth factor.

50. A method of therapeutically treating a degenerating intervertebral disc, comprising the steps of:
a) determining a level of a pro-inflammatory protein within the disc,
b) comparing the level against a pre-determined level of the pro-inflammatory protein, and
c) injecting a high specificity p38 kinase inhibitor into the disc.

51. The method of claim 50 wherein the proinflammatory protein is an interleukin.

52. The method of claim 51 wherein the predetermined level for the interleukin is at least 100 pg/ml.

53. The method of claim 50 wherein the proinflammatory protein is an interleukin-6.

54. The method of claim 53 wherein the predetermined level for the interleukin-6 is at least 100 pg/ml.

55. The method of claim 53 wherein the predetermined level for the interleukin-6 is at least 250 pg/ml.

56. The method of claim 50 wherein the proinflammatory protein is an interleukin-8.

57. The method of claim 56 wherein the predetermined level for the interleukin-8 is at least 500 pg/ml.

58. The method of claim 50 wherein the proinflammatory protein is PGE2.

59. The method of claim 58 wherein the predetermined level for PGE2 is at least 1000 pg/ml.

60. The method of claim 50 wherein the proinflammatory protein is TNF-α.

61. The method of claim 60 wherein the predetermined level for TNF-α is at least 20 pg/ml.

62. The method of claim 60 wherein the predetermined level for TNF-α is at least 30 pg/ml.

63. The method of claim 50 wherein the predetermined level for TNF-α is at least 1000 pg/disc.

64. A method of preventing degeneration of an intervertebral disc in a human individual, comprising:
a) determining a genetic profile of the individual,
b) comparing the profile of the individual against a pre-determined genetic profile level of at-risk humans,
c) determining that the individual is at at-risk patient, and
d) injecting a formulation comprising an effective amount of a high specificity p38 kinase inhibitor into a disc of the individual.

65. A method of treating degenerative disc disease in an intervertebral disc having a nucleus pulposus, comprising transdiscally administering an effective amount of a formulation comprising a high specificity antagonist of COX-2 enzyme into an intervertebral disc.

66. A method of treating degenerative disc disease in an intervertebral disc having a nucleus pulposus, comprising transdiscally administering an effective amount of a formulation comprising a high specificity antagonist of NO synthase into an intervertebral disc.

67. The method of claim 66 wherein the high specificity antagonist of NO synthase is selected from the group consisting of N-iminoethyl-L-lysine (L-NIL), and N^{G}-monomethyl-L-arginine.

68. A method of treating degenerative disc disease in an intervertebral disc having a nucleus pulposus, comprising transdiscally administering an effective amount of a formulation comprising a high specificity antioxidant into an intervertebral disc.

69. A method of treating degenerative disc disease in an intervertebral disc having a nucleus pulposus, comprising transdiscally administering an effective amount of a formulation comprising a high specificity anti-proliferative agent into an intervertebral disc.

70. The method of claim 69 wherein the high specificity anti-proliferative agent is rapamycin.

71. The method of claim 70 wherein the rapamycin is provided in an about 0.1 to about 10 μM dose.

72. The method of claim 69 wherein the high specificity anti-proliferative agent is a cdk inhibitor.

73. The method of claim 72 wherein the cdk inhibitor is provided in an about 0.1 to about 10 μM dose.

74. A method of treating degenerative disc disease in an intervertebral disc having a nucleus pulposus, comprising transdiscally administering an effective amount of a formulation comprising a high specificity anti-apoptotic agent into an intervertebral disc.

75. The method of claim 54 wherein the anti-apoptotic agent is selected from the group consisting of EPO, erythropoetin mimetic peptides, EPO mimetibodies, IGF-I, IGF-II, and caspase inhibitors.
